(19) United States
(12) Patent Application Publication
(10) Pub. No.: US 2026/0345075 A1
(43) Pub. Date: Nov. 5, 2026

(54) WEARABLE DEVICE AND METHOD FOR MONITORING BIOLOGICAL CHARACTERISTICS

(71) Applicant: FOCAL LINES TECHNOLOGIES INC., Vancouver (CA)

(72) Inventors: Quoc Sy VU, Vancouver (CA); Linh LE, Vancouver (CA)

(21) Appl. No.: 19/485,270

(22) PCT Filed: Feb. 28, 2024
(86) PCT No.: PCT/CA2024/050251

§ 371 Date: Nov. 17, 2025

(30) Foreign Application Priority Data
May 19, 2023 (CA) 3200058

(51) Int. Cl. A61B 5/00; A61B 5/256; A61B 5/263; A61B 5/274; A61B 5/296

(52) U.S. Cl. CPC A61B 5/6804; A61B 5/256; A61B 5/263; A61B 5/274; A61B 5/296

(57) ABSTRACT

A system and method for monitoring of biological characteristics are disclosed. The system includes a plurality of electrically conductive electrodes; and a wearable textile having: embedded electrical pathways for electrically connecting the plurality of electrically conductive electrodes; and a plurality of electrically conductive connector pads for removably fixing the electrodes to the wearable textile. A method for monitoring biological characteristics of a user, utilizes the apparatus and involves: attaching the electrodes to the connector pads; having the wearable textile worn by a user such that the first and second electrodes are adjacent the user's skin; receiving signals from sensors in electrical communication with said electrically conductive electrodes in a signal processing system; and processing the received signals to output data representative of the biological characteristics of the user.

### TECHNICAL FIELD

The specification generally relates to a system and a method for measuring and monitoring biological characteristics. The specification also relates to a wearable device and a method for monitoring biological characteristics.

### BACKGROUND

It is generally beneficial to measure and monitor various biological characteristics of individual persons or animals in connection with maintenance of their health. These measurements permit one to better understand the health status of the subjects being monitored and to predict trends over time. Traditionally, the required apparatus for performing such monitoring was typically bulky and expensive, often requiring specialized skills or training to utilize.

For at least the foregoing reason, biological characteristics would normally only be tracked when the resources were available for use. Accordingly, monitoring and tracking of important biological functions including vital signs were conducted when the subject was in a suitable setting such as in a hospital, at a doctor's office, or while a testing facility such as a medical a laboratory. Unfortunately, this often led to situations where such monitoring could not take place in the ordinary course of general activities such as walking, sleeping, or during physical exercise.

Recent advances in sensor technology have resulted in the availability of specialized sensors that can be integrated into special purpose devices and accessories. For example, belts may be made available to track a user's heart rate at the gym, and certain smart watches include integrated sensors for monitoring biological characteristics that are derivable from the wearer's wrist. Similarly, smart shirts can be designed with integrated sensors to track specific biological characteristics at defined locations.

However, such existing technologies generally provide limited functionality and are inflexible. Although wearable devices that measure specific biological characteristics are known, improvements in one or more of: flexibility, customizability, ease of use, accuracy and cost are desired to help mitigate the aforementioned challenges.

### SUMMARY OF THE DISCLOSURE

The present disclosure relates to a wearable electromyogram (EMG) monitoring apparatus using graphene-textile based contact sensors. Graphene-based sensors are designed to satisfy user comfort and to support long-term monitoring applications. The wearable apparatus is built to collect the EMG signal from muscles in the body by using graphene electrodes which can be integrated into clothing. The apparatus supports long-term monitoring without undue inconvenience or unpleasantness for users.

In accordance with an aspect of the present disclosure, there is provided a method for monitoring biological characteristics using an apparatus. The apparatus comprises: (i) a wearable textile having a first and second embedded electrical pathways; and (ii) a first connector pad and a second connector pad disposed in electrical communication with the first and second electrical pathways respectively and a plurality of electrically conductive electrodes comprising a first electrode and a second electrode. The method comprises: (i) attaching the first electrode to the first connector pad; (ii) attaching the second electrode to the second connector pad; (iii) having the wearable textile worn by a user, such that the first and second electrodes are adjacent the user's skin; (iv) receiving signals from sensors in electrical communication with said electrically conductive electrodes in a signal processing system; and (v) processing the signals to output data representative of the biological characteristics of the user.

The biological characteristic may be an electromyogram (EMG), an EKG, or breathing rate, for example.

The method may further comprise mating each fixation structure to one of the plurality of connector pads. The method may further comprise selectively attaching the first set of electrically conductive electrodes to a first set of the plurality of connector pads, to measure a first category of measurements, and applying the second set of electrically conductive electrodes, different from the first set of electrically conductive electrodes, to a second set of the plurality of connector pads, to measure a second category of measurements different from the first category of measurements.

In accordance with another aspect of the present disclosure, the sensors may be made of an electrically conductive material such as a graphene oxide/polymer sensor, MXene/polymer or another semiconductor sensing material to detect muscle signal. The signal may be acquired by placing graphene electrodes on the skin surface of a specified target muscle. The electrode placement may follow EMG measurement convention for obtaining a good signal. A first electrode, namely “MID”, may then be placed on the middle of the target muscle. A second electrode, namely “END”, may then be placed adjacent the middle electrode towards an end of the muscle body. A third electrode, namely “REF”, may then be placed on a bony or nonadjacent muscular part of the subject's body near the targeted muscle. The signal obtained from the skin surface is a differential signal created by the difference between the signal from the MID electrode and the END electrode.

Other technical advantages may become readily apparent to one of ordinary skill in the art after review of the following figures and description.

### BRIEF DESCRIPTIONS OF THE DRAWINGS

For a better understanding of the embodiments described herein and to show more clearly how the embodiments may be carried into effect, reference will now be made, by way of example only, to the accompanying drawings in which:

FIG. 1 shows the placement of electrodes of an apparatus for wearable monitoring of a user, in one embodiment of the present disclosure;

FIG. 2 shows the embodiment of FIG. 1 when worn by the user;

FIG. 3 shows an embodiment of a system that includes an apparatus for wearable monitoring, a signal acquisition unit, and a smart phone;

FIG. 4 is a perspective view of a plurality of electrodes for use in an embodiment of an apparatus for wearable monitoring;

FIG. 5 is a schematic diagram of an electrode depicted in FIG. 4, in cross section, for use in an embodiment of an apparatus for wearable monitoring;

FIG. 6 shows another embodiment of an apparatus for wearable monitoring suitable for use with a shoe;

FIG. 7 shows the embodiment FIG. 6 in the context of a sneaker;

FIG. 8 is a block diagram showing a system for processing signals from an apparatus for wearable monitoring in another embodiment of the present disclosure;

FIG. 9 is a schematic diagram of a system in accordance with this disclosure;

FIG. 10 illustrates a process for preparation of electrodes for use in a system in accordance with this disclosure;

FIGS. 11A and 11B illustrate the preparation of fabric for use as an electrode in a system in accordance with this disclosure; and

FIG. 12 shows schematically the preparation of electrodes for use in a system in accordance with this disclosure.

Unless otherwise specifically noted, articles depicted in the drawings are not necessarily drawn to scale.

### DETAILED DESCRIPTION

Directional terms such as “top,” “bottom,” “upwards,” “downwards,” “vertically,” and “laterally” are used in the following description for the purpose of providing relative reference only, and are not intended to suggest any limitations on how any article is to be positioned during use, or to be mounted in an assembly or relative to an environment. The use of the word “a” or “an” when used herein in conjunction with the term “comprising” may mean “one,” but it is also consistent with the meaning of “one or more,” “at least one” and “one or more than one.” Any element expressed in the singular form also encompasses its plural form. Any element expressed in the plural form also encompasses its singular form. The term “plurality” as used herein means more than one, for example, two or more, three or more, four or more, and the like.

In this disclosure, the terms “comprising”, “having”, “including”, and “containing”, and grammatical variations thereof, are inclusive or open-ended and do not exclude additional, un-recited elements and/or method steps. The term “consisting essentially of” when used herein in connection with a composition, use or method, denotes that additional elements, method steps or both additional elements and method steps may be present, but that these additions do not materially affect the manner in which the recited composition, method, or use functions. The term “consisting of” when used herein in connection with a composition, use, or method, excludes the presence of additional elements and/or method steps.

For clarity and where considered appropriate, reference numerals may be repeated among the figures to indicate corresponding or analogous elements. In addition, numerous specific details are set forth in order to provide a thorough understanding of the embodiment or embodiments described herein. In other instances, well-known methods, procedures and components have not been described in detail so as not to obscure the embodiments described herein. It should be understood at the outset that, although embodiments are illustrated in the figures and described below, the principles of the present disclosure may be implemented using any number of techniques. The present disclosure should in no way be limited to the implementations and techniques illustrated in the drawings and described in this disclosure.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or as a non-limiting example, and not necessarily as “preferred” over other embodiments. Further definitions for terms may be set out herein; these may apply to prior and subsequent instances of those terms, as will be understood from a reading of the present description. It will also be noted that the use of the term “a” or “an” will be understood to denote “at least one” in all instances unless explicitly stated otherwise or unless it would be understood to be obvious that it must mean “one”.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

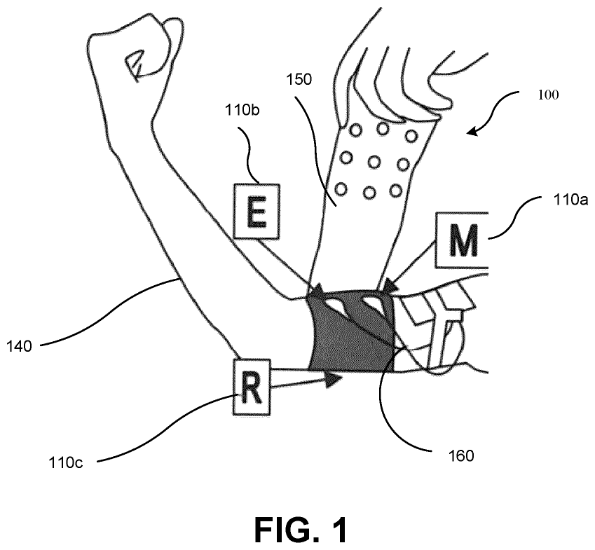

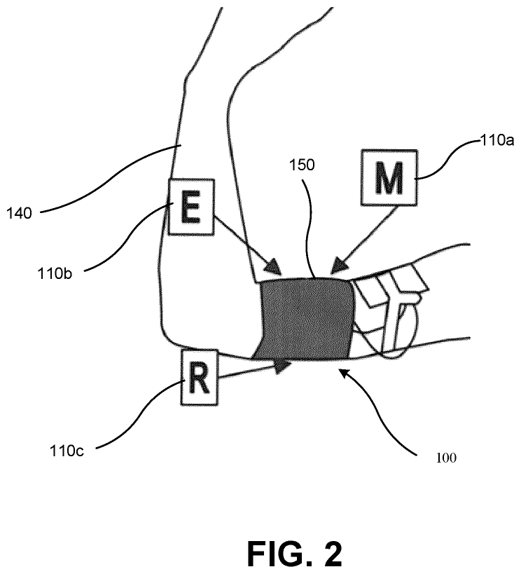

Referring to FIGS. 1 and 2, and according to an embodiment of a wearable apparatus, there is a wearable apparatus 100 for monitoring biological characteristics of a user being worn. The apparatus 100 comprises a plurality of electrodes in the form of electrodes 110a, 110b, and 110c (individually and collectively “electrodes 110”), and a wearable textile material in the form of an armband 150. The armband 150 is intended to be worn on an arm 140 of a subject (e.g., human). In other embodiments, the textile material can take the form of other articles of clothing, such as socks, compression sleeves, shorts, bodysuits or components of a shoe such as insoles. The electrodes 110 are electrically conductive and may be formed, for example, from graphene oxide or MXene materials, as well as more traditional materials.

The wearable textile material is provided with embedded electrical pathways. Accordingly, armband 150 is provided with electrical pathways 160 for electrically connecting the plurality of electrically conductive electrodes 110 to an acquisition circuit (not shown in FIG. 1).

FIG. 2 depicts the apparatus 100 of FIG. 1 with the armband 150 as fully worn by the user or subject 140. In some embodiments, the wearable textile material of armband 150 is further provided with a plurality of electrically conductive pads (not specifically illustrated in FIG. 1 or FIG. 2) to which at least a first electrically conductive electrode 110a and a second electrically conductive electrode 110b are removably attached.

As the electrically conductive electrodes 110 are attached to the wearable textile material in armband 150 that is worn by user 140, the first and second electrically conductive electrodes 110 are disposed proximate, adjacent or abutting the user's skin and are able to detect a biological characteristic of the user 140.

The biological characteristics that are measured and monitored by apparatus 100 may, for example, include Electromyography (EMG) readings from a muscle of a user 140, electrocardiogram (EKG) readings from a user's heart, electroencephalogram (EEG) readings of a user's brain activity, breathing rate, or other vital signs that can be measured superficially by way of appropriate electrodes. In the embodiments described herein, the biological characteristics may be discussed in terms of EMG readings, but it will be understood that other measurements may be taken with minor modifications.

By way of background, prior to the exertion of force by a given muscle, small electrical currents are generated by muscle fibres, a part of the signaling process for the muscle fibres to contract. The signal is known as the electromyogram (EMG) can be measured by applying electrodes to the skin surface.

EMG signal amplitude can vary from microvolts to millivolts and may depend on various factors such as timing and intensity of muscle contraction, distance of electrode from the active muscle area, electrode and amplifier circuit properties, quality of contact to the skin and the like.

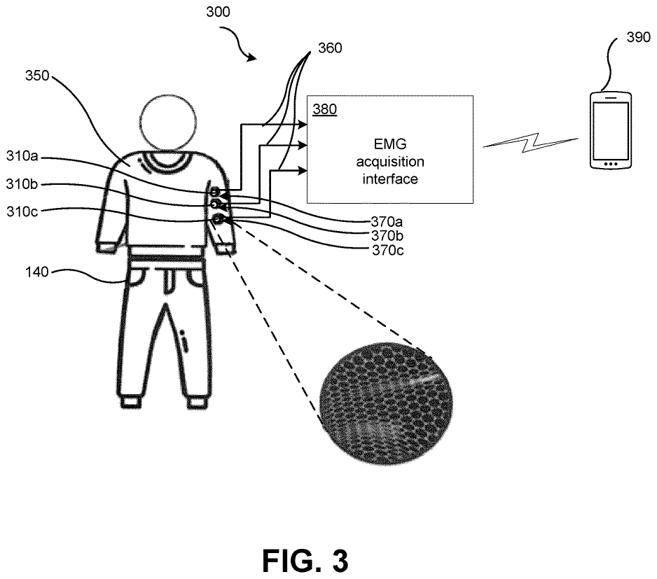

According to another embodiment of a wearable apparatus, and referring to FIG. 3, there is a wearable apparatus 300 for monitoring of biological characteristics. The apparatus 300 comprises: (i) a plurality of electrodes in the form of electrodes 310a, 310b, and 310c (individually and collectively “electrodes 310”); and (ii) a wearable textile material in the form of a shirt 350 to be worn by user 140. In other embodiments, the textile material can take the form of other articles of clothing, such as socks, compression sleeves, shoe components, such as insoles, shorts, or bodysuits.

As contemplated in this embodiment, electrically conductive electrodes such as electrodes 310 are formed from graphene oxide or MXene materials. In other embodiments, electrodes can be formed from traditional materials. Apparatus 300 further comprises the wearable textile material in the form of shirt 350 worn by the user 340 in order to properly position the electrodes 310.

As contemplated in this embodiment, the wearable textile material in the form of shirt 350 is provided with embedded electrical pathways. Accordingly, shirt 350 comprises electrical pathways 360 for electrically connecting the plurality of electrically conductive electrodes 310 to an acquisition interface circuit 380.

In some embodiments, the wearable textile material 350 is further provided with a plurality of electrically conductive connector pads 370a, 370b, 370c (individually and collectively “connector pads 370”) to which a first electrically conductive electrode 310a, a second electrically conductive electrode 310b and a third electrically conductive electrode 310c and are removably attached.

As the electrically conductive electrodes 310 are attached to the wearable textile material 350 in the form of a shirt worn by a user, via connector pads 370, the electrically conductive electrodes 310 are disposed proximate, adjacent the user's skin and are able to detect a biological characteristic of the user 140.

In some embodiments, only two electrodes (e.g., electrodes 310a, 310b) are used in order to detect a biological characteristic across two locations on a user's skin. For example, the first electrode 310a may be placed or positioned by the wearable textile material of shirt 350 adjacent a middle of a selected muscle. The second electrode 310b may then be positioned by virtue of its location on the wearable textile forming the shirt 350, adjacent an end of the same selected muscle. Such a configuration can allow the electrodes 310 to acquire an EMG measurement, for example. The embedded electrical pathways 360 electrically connect the first electrode 310a and the second electrode 310b. The wearable textile then further include an interface or acquisition interface circuit 380 for outputting measurements based on at least the first and second electrodes 310a and 310b. The electrical pathways 360 may comprise or partially comprise EMG cables for connecting the electrodes 310.

In some embodiments, a third electrode 310c may be provided and located by the wearable textile of shirt 350 adjacent a body part spaced apart from the selected muscle. The third electrode 310c may function as a reference electrode, and may be placed on and located adjacent a bone or another muscle spaced apart from the targeted muscle.

In some embodiments where the measurement sought is an EMG signal, electrodes 310 are placed on the skin surface of a specified muscle to obtain the signal. The electrode placement may follow EMG measurement convention for obtaining a good signal. In an embodiment, the first electrode 310a, namely MID, is placed on the middle of the desired muscle. The second electrode 310b, namely END, is placed adjacent to the middle electrode towards the end of the muscle body. The third electrode 310c, namely REF, is placed on a bony or nonadjacent muscular part of user's body near the targeted muscle. In some embodiments, it is contemplated that the third electrode can be used to reduce 50/60 Hz interference from surroundings such as, but not limited to, those interferences emitted from nearby power lines.

The signal obtained from the skin surface in this manner is a differential signal created by the difference between the signal from the first electrode 310a and the second electrode 310b. The MID and END electrodes 310a, 310b would then be used together in order to get the EMG signal. The REF electrode 310c then acts independently of the MID & END electrodes 310a, 310b. As discussed in more detail below, the apparatus 300 can incorporate a plurality of electrode pairs for the purpose of obtaining EMG signals.

In some embodiments, the acquisition interface circuit 380 comprises a base unit for processing and outputting any data acquired from the electrodes 310. Such output may be provided by a wireless connection to a portable device 390 as depicted in FIG. 3. In other embodiments, a wired connection can be provided. In other embodiments, the base unit can store data acquired and such data can be offloaded to a user device, such as a smartphone or a computer after concluding a measurement period. Data from different components of apparatus 300 can be transmitted by a wired or wireless means. In some embodiments, the base unit can be removed from the wearable textile 350 and separately connected it to a user device 390.

In other embodiments, the acquisition interface circuit 380 can be a docking location incorporated into the wearable textile 350 for locating a base unit. The docking location may then be configured to place the base unit in electrical contact with at least one of the embedded electrical pathways 360, thereby integrating the base unit into a data acquisition circuit.

In some embodiments where data is output by acquisition interface circuit 380 or by a base unit associated with the interface, the signal generated may be processed, analyzed, and displayed on common consumer electronic devices, such as laptops, smartphones, and smart watches.

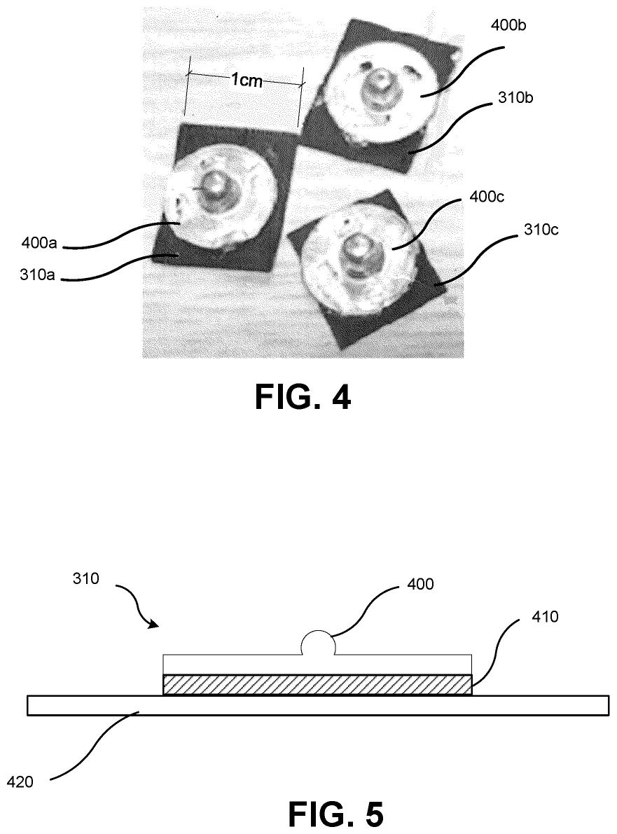

FIG. 4 depicts a perspective view of electrodes 310a, 310b and 310c in a form suitable for incorporation into a wearable apparatus.

FIG. 5 is a cross-sectional schematic diagram of one of the electrodes 310. As shown in FIG. 5, the electrically conductive electrodes 310 each comprise a corresponding fixation structure 400 (individually and collectively “fixation structures 400”) for mating with corresponding connector pads 370a, 370b, 370c of the wearable textile 350.

The combination of the fixation structures 400 with electrically conductive electrodes 310 and the connector pad 370 of the wearable textile 350 form a snap type closure. In such an embodiment, the fixation structures 400 of the electrodes 310 may be a male snap button as shown, or it may be a female snap button. Accordingly, the connector pads 370 of the wearable textile 350 may be the one of male or female snap components not used on the electrodes 310, such that it can mate with the fixation structures 400 of the electrodes. For example, the female snap button may be sewn into the wearable textile 350 for use as connector pad 370.

As contemplated in this embodiment, the connector pads 370, and fixation structures 400 are electrically conductive, and it is further contemplated that the electrodes 310 can be placed in electrical connection with corresponding pathways 360. Accordingly, a conductive element 420 of the electrode can be formed from an appropriate material, such as but not limited to graphene oxide. Fixation structure 400 can be in the form of a metallic button, and can be fixed to the graphene oxide by silver-epoxy 410 or other suitable conductive adhesive. In some embodiments, the graphene oxide electrode can incorporate a polymer substrate. In other embodiments, the snap can be fixed directly to the graphene oxide by, for example, riveting.

In some embodiments, the graphene oxide conductive element 420 is a sensor. In other embodiments, instead of metallic snaps, the fixation structure 400 is an elastic textile.

In the embodiment shown, the wearable textile 350 has three connector pads 370a, 370b and 370c which mate with three corresponding fixation structures 400a, 400b and 400c of the electrodes 310a, 310b and 310c respectively. However, in some embodiments, the plurality of electrically conductive electrodes can comprise a first set of electrically conductive electrodes, such as those shown in FIG. 4, and a second set of electrically conductive electrodes (not shown). Each electrode of the first set may then have a distinct electrode structure from each electrode of the second set. For example, each electrode of the first electrode set may be distinct from each electrode of the second electrode set in terms of size, supporting fabric, electrode material, and other physical characteristics. As noted above, the apparatus 300 described herein can be used to take different types of measurements of different biological characteristics. Accordingly, the first set of electrodes may be sized or otherwise configured for taking an EMG measurement, while the second set of electrodes may be sized or otherwise configured for taking EKG measurements.

Further, while the embodiments discussed herein are discussed in terms of electrodes, various sensor implementations may be used as well. Accordingly, one of the plurality of sets discussed may include more sophisticated sensor structures that can connect to the same connector pad 370 used for the electrodes 310 shown.

Each electrode of each set is provided with corresponding fixation structures 400, and as such, when multiple sets are provided, electrodes from any one such set may be applied to the fixation structures of the wearable textile 350. Accordingly, a user 140 can selectively apply first electrode 310a and second electrode 310b from one of the sets of electrodes to the connector pad 370 of the wearable textile 350 based on a specific category of measurement sought by the user.

Similarly, while the wearable textile 350 may have three connector pad 370, more fixation structures may be provided. In some embodiments, the electrodes 310 can then be removably fixed to a subset of the connector pad 370 of the wearable textile 350. For example, the subset of the electrically conductive connector pad 370 of the wearable textile 350 may be selected based on user 140 anatomy so as to position the electrically conductive electrodes 310 for best detecting the desired biological characteristic.

Accordingly, a user may select appropriate connector pad 370 based on the size of their biceps the length of their arm, or the particular type of measurement being taken.

In other embodiments, connector pad 370 can be provided at different locations within the wearable textile 350. Where the wearable textile 350 is a shirt, as shown in FIG. 3, and each sleeve may have a set of connector pad 370 such that a user 140 may select which fixation structures to use based on which body part is to be monitored by the apparatus 300. Similarly, a shirt may have fixation structures at the user's chest, or at a user's lower back, and shorts or pants may be provided with fixation structures at each leg. Similarly, a full body suit may be provided with fixation structures along both arms and legs, as well as additional locations. All such fixation structures may then be in electrical communication with the electrical pathways within the wearable textile 350.

In some embodiments, covers may be provided for connector pad 370 not in use, so as to electrically insulate such fixation structures from the user's skin, and so as not to provide noise in the electrical signals being analyzed.

In some embodiments, the apparatus 300 is configured so as to compress the electrodes 310 against the user's 140 skin with some force. This may be, for example, by providing at least a portion of the wearable textile 350 of an elastic material. This may be by utilizing an elastic material for the entire wearable textile 350 such that it forms a pressure sleeve or an elastic shirt, or it may be by providing an elastic material just for the portion of the wearable textile 350 to be used for taking measurements, such as the sleeves or legs of a shirt or pants respectively.

In some embodiments, the wearable textile is inelastic, but a secondary textile is provided to apply compression to the wearable textile. For example, where the wearable textile is an inelastic shirt, a compression fabric may be provided for wearing over the shirt, such that a user 140 can apply the compression fabric to only the portion of the shirt being used for taking measurements. Accordingly, the wearable textile may be polyester, nylon or cotton.

In some embodiments, electrodes 310 can be sewn directly into the fabric. Such fabric can have an elastic element in order to keep the electrodes in contact with the skin of the user. Such an armband may be designed to wrap tightly around the subjects arm, for example. Alternatively, a thin fabric layer may be used to cover the outside of the electrode apparatus to keep the systems table when the user is performing activity.

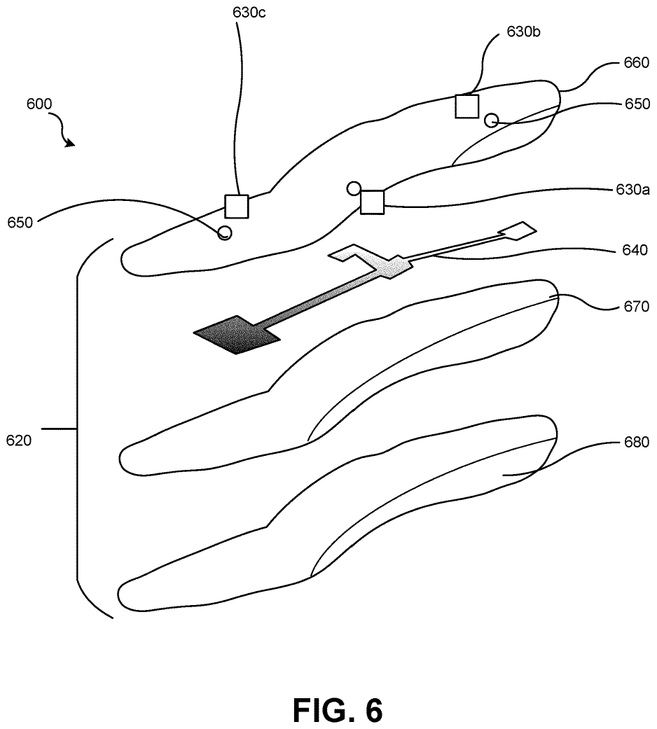

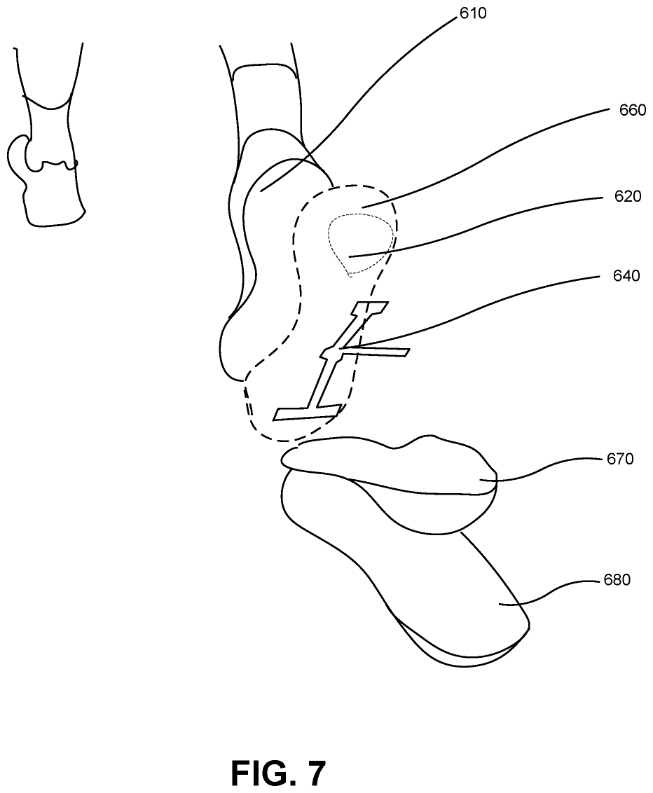

FIG. 6 shows an exploded view of yet another embodiment of the wearable apparatus in the form of a wearable apparatus 600 for monitoring biological characteristics. FIG. 7 shows this embodiment of the apparatus 600 in the context of a sneaker 610.

As contemplated in this embodiment, the wearable textile material is an insole 620 provided with a plurality of electrically conductive electrodes 630a, 630b, 630c (individually and collectively “electrodes 630”). The insole 620 can be embedded with electrical pathways 640 for electrically connecting the electrically conductive electrodes 630.

The insole 620 is provided with electrically conductive connector pads 650 which place any electrode 630 fixed therein in electrical connection with the corresponding electrical pathways 640. When the electrically conductive electrodes 630 are fixed to the wearable textile material in the form of insole 620 and when the wearable textile material is worn by a user 140, the electrically conductive electrodes are located adjacent the user's skin for detecting a biological characteristic of the user.

In some embodiments, the insoles 620 shown are worn without socks such that the electrodes 630 are adjacent the user's skin. In other embodiments, an insole structure may be integrated into a pair of socks to be used for the evaluation described herein.

As contemplated in this embodiment, the insole 620 comprises a plurality of layers 660, 670, 680, which may be formed from a single textile material or from distinct materials. When formed from a single material, the insole 620 may be a single unit with the electrical pathways 640 embedded within and with the connector pads 650 emerging at a top surface. When formed from multiple materials, or multiple layers of the same material, the electrical pathways 640 may be embedded between layers 660, 670, 680 of the insole 620, as shown, and the connector pads 650 may extend through the top layer 660. For example, the insole 620 may comprise a lower structural or stiffening layer 680, a central cushioning layer 670, and a top layer 660 designed to be in contact with a user's foot.

As shown, in some embodiments, the first and second electrodes 630a, 630b, when fixed to insole 620, may be located so as to be adjacent the middle of and the end of a muscle of a user respectively when the insole 620 is incorporated into a sneaker 610 and worn. A third electrode 630c may then be a reference electrode located to be adjacent a bone or unrelated muscle.

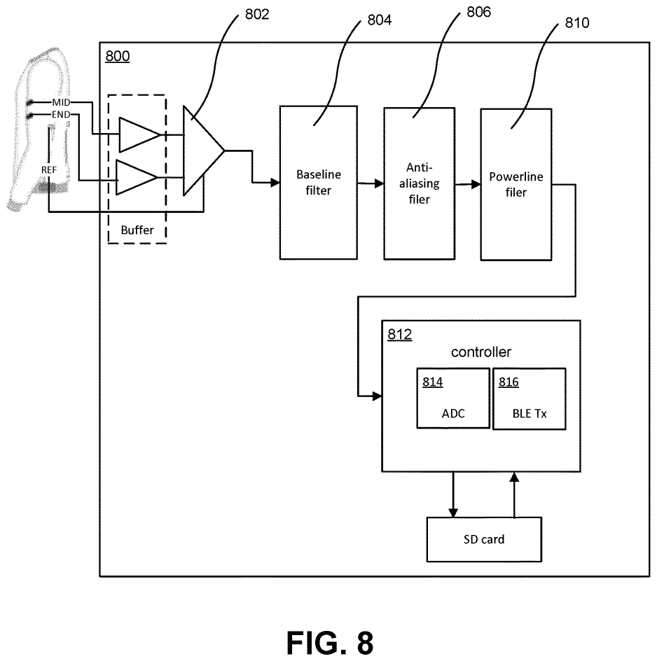

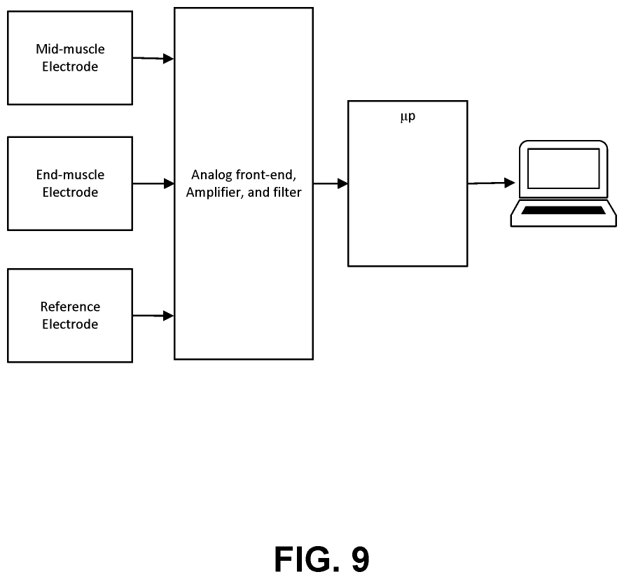

FIG. 8 is a block diagram showing a system for processing signals from a wearable apparatus for monitoring biological characteristics. FIG. 9 is a schematic diagram of a system in accordance with an aspect of this disclosure.

The embodiments described herein provide various benefits relative to earlier implementations, many of which have already been discussed. For example, the communication components shown in FIGS. 8 and 9 allow the graphene sensor to be connected to the system, which is designed to be simple and suitable for integration on clothing or on the body. The system of iron buttons is used as a system of male and female connectors to communicate between the graphene sensor and the signal processing electronic block.

Another difference of the system is the potential for integrated signal processing electronic block 800 including filter circuits, amplifier circuits that allow for custom gain, and Bluetooth signal transceiver system combined with microchip processing that allows the signals derived from the electrodes to be sent directly to the user's smartphone via Bluetooth.

An additional difference of the system is that the application software on Android™ and iOS® operating systems displays signals and signal-processed parameters for users to view and monitor in real time. In the absence of a Bluetooth connection, the generated signal can be saved to the included SD card.

An additional difference of the system is that the application on Android™ and iOS® integrates machine learning technology to detect chronic diseases of the muscles through the analysis of electromyographic signals. When a signal indicative of a disease is detected, the system can send alarm signals to designated doctors via SMS or IP-based technology.

Following the signal path, two buffers are used to reduce the interference that may enter the system due to the impedance mismatch between two differential electrodes. The buffered signal is considered as a differential signal and is amplified by the instrumentation amplifier.

The amplifier 802 comprises the INA 333 low-power precision instrumentation amplifier from Texas Instruments which has a high common mode rejection ratio (CMRR)—rated at 100 dB minimum. The amplifier gain may be set at 101. The output of the INA 333 may be followed by a first order active high pass filter (HPF) 804 at 30 Hz to mitigate any remaining low frequency noise such as motion artifacts as well as any potential DC offset from INA.

In the embodiment shown, the HPF is followed by a first order low pass filter (LPF) at 500 Hz worked as an antialiasing filter 806. After that, the output signal is filtered by the power line interference filter 810 to eliminate power line noise.

For the purpose of saving power for a wearable system, in some embodiments, the system uses Bluetooth Low Energy module 816 to transfer the data to a smartphone. In some specific embodiments, BLE module NRF 52832 from Nordic Semiconductor may be used because of the common use and full assistance.

A controller 812 includes analog-digital converter (ADC) 814 and Bluetooth Low Energy module 816. As contemplated herein, the ADC 814 is used to sample the EMG signal from the analog stage and digitized. Each sample may be represented using 12 bits per sample, and the analog signal may be sampled at a sampling rate of 1000 Hz or other rates that satisfy the Nyquist criterion.

The digitized data from the electronic circuit may then be transferred to the smartphone via BLE connection. In some embodiments, an Android™ application was developed to receive and process the EMG data. This smartphone program allows users to see the signal run real time on the screen and save data into text files in a computing device such as a smartphone memory for further analysis.

Accordingly, a power line notch digital filter and Fast Fourier Transform (FFT) may also integrated into the app for signal preprocessing.

Users are then able to see a real-time EMG signal that can show muscle contraction for the purpose of fitness monitoring for the calf and the bicep. Users are able to see muscle contraction of the lower back and the foot for the purpose of monitoring a medical condition.

For data visualization and further processing, an Android™ smartphone and/or iOS® app may be developed. The EMG monitoring application may include data transmission with BLE (Bluetooth Low Energy), data storage and signal processing algorithms. The measured EMG signals are evaluated for amplitude, signal-to-noise ratio, correlation coefficient, peak frequency and intensity spectrum obtained through Fast Fourier Transform (FFT) math.

The first time the user accesses the application, it needs to grant access to the internal memory to save data and allow the phone's Bluetooth to be turned on. Before performing a signal measurement, the user may open the menu of the application and add personal information so that the device can be saved with the measurement data later.

FIGS. 11A and 11B illustrate the preparation of fabric 420 for use as an electrode in a system in accordance with this disclosure. FIG. 12 shows schematically the preparation of electrodes 310 for use in a system 300 in accordance with this disclosure.

In some embodiments, the following materials and method were used in order to prepare coated nylon filaments with graphene oxide (rGO). Commercially available high-purity aqueous solution of graphene oxide (GO) solution (named SLGO 300-800 nm, 2 mg/ml) nylon filaments (>99% polyamide, diameter 500 m) and polyester fabric (>99% polyester) were used. The nylon filaments provided a chemically stable and smooth substrate surface while being highly flexible and stretchable. The polyester fabric provided excellent flexibility along with good heat conductivity and electrical insulation. The rGO-coated textile (abbreviated as rGO-textile) was prepared by dipping the woven cotton textile into GO (2 mg/ml) solution. The GO solution would be dried out, and the GO will be thermally reduced at 160 °C for 6 hours.

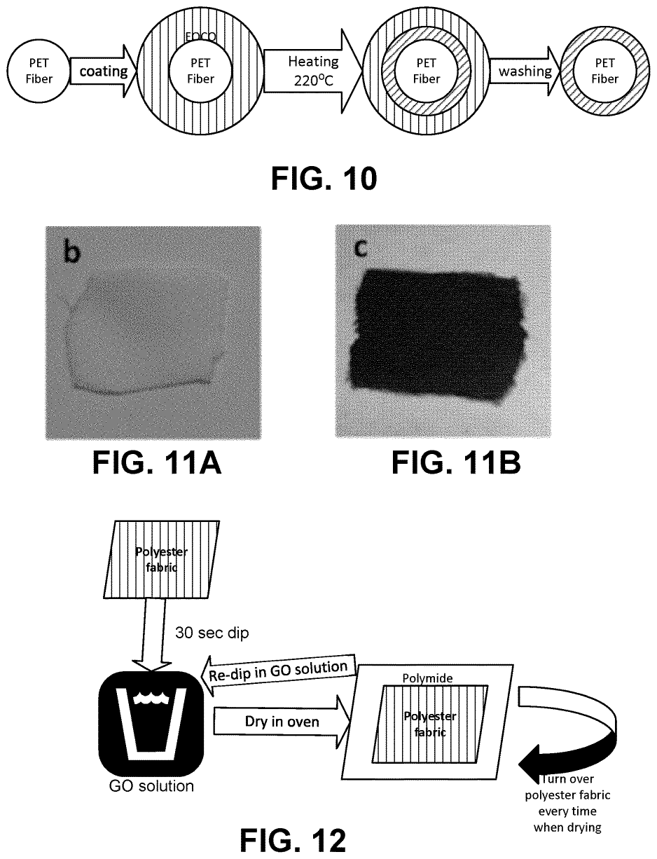

In some embodiments, edge-oxidized graphene oxide in H₂O (2 mg/ml) was placed in an ultrasonic bath for 30 min and was drop-casted onto 2×2 cm2 PET fabric. One mL 0.2 wt % edge-oxidized graphene oxide solution was dispersed per cm2 of PET fabric and allowed to dry at 90 °C between each coating on a hotplate. After the final coating, the EOGO-PET was heat-treated at 220 °C for 5 seconds to facilitate binding of graphene to the outer fibers as shown in FIGS. 11A-B. Conductivity was first observed after the second coat and the resistance decreased significantly thereafter. After several iterations of a coating process, the resistance density decreased. The optimal number of coatings for sensors was evaluated, and in some embodiments, such an optimal number was 10, resulting in uniformly coated fibers as a result of relatively strong π-π interactions formed between the graphene molecule and benzene groups of PET, as shown in FIGS. 10 and 11A. The pre-annealed sensor was washed to remove macro-scale agglomerations on the surface and stabilize conductivity. Preliminary resistance measurements are taken using a standard 2-probe method.

To create flexible electrodes, such as those discussed above 310, the prepared graphene coated textile 420 is cut into pieces of an electrode size, such as 1 cm×1 cm to match with the typical electrode size recommended in Surface Electromyography for the Non-Invasive Assessment of Muscles (SENIAM) project of European Union. A snap button, functioning as a fixation element 400 is attached to each graphene piece 420 by Silver-epoxy 410, as discussed above with respect to FIG. 5. Using this configuration, the electronic circuit can easily connect with graphene sensors by EMG cable embedded into the wearable textile 350 as embedded electrical pathways 360.

Accordingly, in some embodiments, a method for preparing an electrically conductive graphene oxide/polymer electrode without external materials is provided, the method comprising the steps of repeatedly dip-coating textiles suspended in an aqueous solution and subsequently drying in air. The solution is then chemically reduced by dipping into an acidic aqueous solution. The solution is then rinsed with distilled water and subsequently dried.

The electrode may then be further processed by compressing the electrically conductive graphene oxide/polymer between two plates and treating by heating in a furnace.

In some embodiments, the textile is polyester. Alternatively, it may also be nylon, cotton, and spandex.

In some embodiments of the process, the textile may be turned over for each drying period.

The aqueous solution may be graphene oxide (GO) or it may be an MXene material.

In some embodiments, the temperature is slowly increased for the heating treatment.

In some embodiments, the resistivity of the electrically conductive graphene oxide/polymer is between 3.59 Kohms and 45.1 Kohms.

The embodiments described herein may incorporate a distinct design for an electromechanical sensor made from graphene. In the alternative to the dipping method described with respect to FIGS. 10-12, the sensor or electrode may be made using graphene printing technology (rGO) and may be integrated onto various textile materials, such as cotton, polyester, and nylon. With the use of a graphene sensor, the received signal is equivalent to that of an Ag/AgCl sensor, a common, single-use sensor on the market. Graphene sensors are designed with user comfort in mind and in applications where long-term monitoring is required. 50 Hz or 60 Hz main line noise and other artifacts are filtered by analog blocks and processed on the digital signal.

While the present disclosure has been described particularity with respect to several specific embodiments, it is not intended that it should be limited to any such particulars or embodiments or any particular embodiment, but it is to be construed with references to the appended claims. Although specific advantages have been enumerated above, various embodiments may include some, none, or all of the enumerated advantages.

Persons skilled in the art will appreciate that there are yet more alternative implementations and modifications possible, and that the above examples are only illustrations of one or more implementations. The scope, therefore, is only to be limited by the claims appended hereto and any amendments made thereto.

What is claimed is:

1. A method for monitoring biological characteristics using an apparatus, wherein the apparatus comprises a wearable textile having a first embedded electrical pathway and a second embedded electrical pathway, a first connector pad of a plurality of connector pads in electrical communication with the first embedded electrical pathway, a second connector pad of the plurality of connector pads in electrical communication with the second electrical pathway, and a plurality of electrically conductive electrodes, the method comprising:
(a) attaching a first electrode of the plurality of electrically conductive electrodes to the first connector pad;
(b) attaching a second electrode the plurality of electrically conductive electrodes to the second connector pad;
(c) positioning the first electrode and the second electrode of the wearable textile adjacent a skin of a subject;
(d) receiving signals from one or more sensors in electrical communication with each of the first electrode and the second electrode in a signal processing system; and
(e) processing the signals to output data representative of the biological characteristics of the subject.

2. The method of claim 1, wherein the biological characteristic is an electromyogram (EMG).

3. The method of claim 1, wherein the plurality of electrically conductive electrodes each comprises a fixation structure for mating with the plurality of connector pads of the wearable textile, the method further comprising mating each fixation structure to one of the plurality of connector pads.

4. The method of claim 3, wherein the plurality of electrically conductive electrodes comprises a first set of electrically conductive electrodes and a second set of electrically conductive electrodes, and wherein the first set and the second set comprise distinct electrode structures, the method further comprising:
(a) selectively attaching the first set of electrically conductive electrodes to a first set of the plurality of connector pads, to measure a first category of measurements; and
(b) applying the second set of electrically conductive electrodes, different from the first set of electrically conductive electrodes, to a second set of the plurality of connector pads, to measure a second category of measurements different from the first category of measurements.

5. The method of claim 4, wherein the first and second category of measurements are selected from the group consisting of EMG, EKG, and breathing rate.

6. The method of claim 3, wherein the fixation structure of each electrically conductive electrode is one of a male or female snap button, and wherein the plurality of connector pads of the wearable textile are the other complementary one of the male or female snap button.

7. The method of claim 3, wherein at least one of the connecting pads comprises an elastic textile.

8. The method of claim 1, wherein the step of attaching the first electrode and the step of attaching the second electrode each further comprises removably fixing the first and second electrodes to the first and second connector pads respectively.

9. The method of claim 8, wherein the first connector pad and the second connector pad of the wearable textile are selected based on an anatomy of the subject so as to position the plurality of electrically conductive electrodes for detecting the biological characteristic.

10. The method of claim 9, wherein the subset of the plurality of electrically conductive fixation structures is selected based on a body part to be monitored by the apparatus.

11. The method of claim 10, wherein the wearable textile is a shirt and the connector pads are disposed in different sleeves of the wearable textile.

12. The method of claim 1, further comprising:
(a) positioning the first electrode adjacent a middle of a selected muscle; and
(b) positioning the second electrode adjacent an end of the selected muscle.

13. The method of claim 12, wherein the wearable textile further comprises a third embedded electrical pathway and a third connector pad disposed in electrical communication with the third embedded electrical pathway, the method further comprising a step of attaching a third electrode to the third connector pad, such that the third electrode is positioned by the wearable textile adjacent a body part spaced apart from a selected muscle.

14. The method of claim 13, wherein the third electrode is located adjacent a bone.

15. The method of claim 1, wherein the wearable textile is an article of clothing, and wherein, when the article of clothing worn by the user, the electrically conductive electrodes are placed adjacent a muscle, positioned for detecting the biological characteristic.

16. The method of claim 1, wherein the wearable textile is at least one of polyester, nylon, or cotton.

17. The method of claim 1, wherein the step of processing the signals comprises one or more of: filtering the received signals to form filtered signals, sampling and digitizing the filtered signal to form digital data; and transmitting the digital data to a computing device for further analysis.

18. The method of claim 17, wherein at least one of the sensors is a graphene based sensor.

19. The method of claim 1, wherein the wearable textile is one of a shirt, an insole and a compression sleeve.

20. The method of claim 1, wherein the wearable textile further comprises a docking location for a base unit, the docking location configured to place the base unit in electrical contact with at least one of the embedded electrical pathways, the method comprising docking the base unit on the docking location.
